(19) United States
(12) Patent Application Publication
(10) Pub. No.: US 2026/0216043 A1
(43) Pub. Date: Jul. 30, 2026

(54) Wax(es)-in-Water Emulsion(s) with a Non-Polar Non-Volatile Hydrocarbon Oil, a C12-C20 Monoalkyl Phosphate Neutralized with a Base, a Fatty Alcohol, a Pullulan, a Latex and a Pigment

(71) Applicant: L'OREAL, PARIS (FR)

(72) Inventors: DELPHINE LAURENT, CHEVILLY-LARUE (FR); BOUCHRA BOUARFA, CHEVILLY-LARUE (FR)

(73) Assignee: L'OREAL, Paris (FR)

(21) Appl. No.: 19/145,595

(22) PCT Filed: Dec. 13, 2023
(86) PCT No.: PCT/EP2023/085665

§ 371 Date: Jul. 3, 2025

(30) Foreign Application Priority Data
Jan. 4, 2023 (FR) FR2300059

(51) Int. Cl. A61K 8/73; A61K 8/06; A61K 8/19; A61K 8/31; A61K 8/34; A61K 8/55; A61K 8/81; A61K 8/92; A61Q 1/10

(52) U.S. Cl. CPC A61K 8/73; A61K 8/062; A61K 8/19; A61K 8/31; A61K 8/342; A61K 8/55; A61K 8/8111; A61K 8/927; A61Q 1/10; A61K 2800/43

(57) ABSTRACT

The present application relates to a composition for caring for and/or making up keratin materials, such as the skin, notably the contour of the eyes, the contour of the eyelashes, the contour of the eyebrows; keratin fibres such as the eyelashes and the eyebrows, in the form of an emulsion, in particular a wax(es)-in-water emulsion, comprising, preferably in a physiologically acceptable medium: a) an aqueous phase, and b) at least one oily phase dispersed in the aqueous phase comprising i) at least one wax; and ii) at least one non-polar non-volatile hydrocarbon oil; and iii) at least one fatty alcohol; and c) at least one C₁₂-C₂₀ monoalkyl phosphate, partially or totally neutralized with a base; and d) at least one pullulan; and e) particles of film-forming polymer suspended in the aqueous phase; and f) at least one pigment.

The present invention relates to the field of caring for and/or making up keratin fibres, and is directed towards providing compositions more particularly intended for making up the eyelashes or eyebrows.

The composition used may notably be in the form of an eyelash product such as a mascara, or an eyebrow product. More preferentially, the invention relates to a mascara. The term “mascara” is understood to mean a composition intended to be applied to the eyelashes: it may be an eyelash makeup composition, an eyelash makeup base (also known as a base coat), a composition to be applied over a mascara, also known as a top coat, or else a composition for the cosmetic treatment of the eyelashes. Mascara is more particularly intended for human eyelashes, but also for false eyelashes.

Compositions for coating keratin fibres of mascara type are generally constituted of at least one fatty phase generally formed from one or more waxes dispersed in an aqueous liquid phase by means of an emulsifying system or carried in an organic solvent. These compositions generally have a particular colouring effect, which is quite often black.

This colouring effect allows users to increase the intensity of the gaze by giving maximum visibility to their eyelashes. The intensity then comes partly from the amount of mascara deposited on the eyelashes and partly from the intensity of the black of the mascara.

The intensity of the black of a mascara formula is then the result of various parameters, essentially resulting from the nature of the pigment used, the content at which it is used and the quality of its dispersion.

However, European regulatory developments will soon tend to restrict the variety of black pigments available for colouring mascaras. It is thus desirable to have a solution which improves the colour intensity of the mascaras, independently of the colouring system used and in particular independently of the pigments used.

Patent FR 2975005B1 proposed mascara compositions in the form of a wax-in-water emulsion comprising at least one particular wax capable of being obtained by, or resulting from, the copolymerization of one or more C₁-C₄ alkene (or olefin) monomers, such as the product with the INCI name ETHYLENE/ACRYLIC ACID COPOLYMER and sold under the reference ASENSA SC 401® by the company HONEYWELL. The example describes a composition comprising at least said wax, the potassium cetyl phosphate anionic surfactant in neutralized form, an aqueous dispersion of an acrylic film-forming polymer (latex), hydroxyethylcellulose and an acacia gum.

Patent application WO 2021249886 has also proposed aqueous mascara compositions comprising an aqueous phase, a waxy phase based on a fatty acid monoester wax having at least 40 carbon atoms, such as behenyl behenate (C₄₄), an anionic surfactant of the fatty acid type neutralized with a base and a semicrystalline polymer.

Mascara compositions in the form of a wax-in-water emulsion comprising waxes, at least one aqueous dispersion of acrylic film-forming polymer (latex), the potassium cetyl phosphate anionic surfactant in neutralized form, pullulan and an acacia gum are known on the mascara market, such as the Fortifying Length & Curl Mascara with Vitamins B3-B6 and Acacia Gum® from Laboratoires Dr Pierre Ricaud.

However, these compositions, after application to keratin fibres such as the eyelashes and the eyebrows, are not completely satisfactory in terms of the coverage and/or the intensity of the colour obtained, in particular black colour.

There is still a need to find new compositions for coating keratin fibres, in particular eyelashes and eyebrows, in the form of a wax-based emulsion which makes it possible to achieve this objective.

Unexpectedly, the inventors have discovered that this objective can be achieved with a composition for caring for and/or making up keratin materials, such as the eyelashes and the eyebrows, in the form of a wax(es)-in-water emulsion, comprising, preferably in a physiologically acceptable medium:
a) an aqueous phase, and
b) an oily phase dispersed in the aqueous phase comprising
i) at least one wax; and
ii) at least one non-polar non-volatile hydrocarbon oil; and
iii) at least one fatty alcohol; and
c) at least one C₁₂-C₂₀ monoalkyl phosphate, partially or completely neutralized with a base; and
d) at least one pullulan; and
e) particles of film-forming polymer suspended in the aqueous phase; and
f) at least one pigment.

This discovery forms the basis of the invention.

### SUBJECTS OF THE INVENTION

Thus, according to one of its aspects, the present invention relates to a composition for caring for and/or making up keratin materials, such as the skin, notably the contour of the eyes, the contour of the eyelashes, the contour of the eyebrows; keratin fibres such as the eyelashes and the eyebrows, in the form of an emulsion, in particular a wax(es)-in-water emulsion, comprising, preferably in a physiologically acceptable medium:
a) an aqueous phase, and
b) at least one oily phase dispersed in the aqueous phase comprising
i) at least one wax; and
ii) at least one non-polar non-volatile hydrocarbon oil; and
iii) at least one fatty alcohol; and
c) at least one C₁₂-C₂₀ monoalkyl phosphate, partially or completely neutralized with a base; and
d) at least one pullulan; and
e) particles of film-forming polymer suspended in the aqueous phase; and
f) at least one pigment.

A second subject of the present invention is a cosmetic method for caring for and/or making up human keratin materials, such as the skin, notably the contour of the eyes, the contour of the eyelashes, the contour of the eyebrows; keratin fibres such as the eyelashes and the eyebrows, consisting in applying to said keratin materials a composition as defined above.

### Definitions

In the context of the present invention, the term “keratin material” is notably intended to mean the skin, notably the contour of the eyes, the contour of the eyelashes, the contour of the eyebrows; keratin fibres such as the eyelashes and the eyebrows. For the purposes of the present invention, this term “keratin fibres” also extends to synthetic false eyelashes.

The term “physiologically acceptable” is understood to mean compatible with the skin and/or its superficial body growths, which exhibits a pleasant colour, odour and feel and which does not cause unacceptable discomfort (stinging, tautness) liable to dissuade the consumer from using this composition.

For the purposes of the invention, the term “emulsion” means any composition comprising a continuous aqueous phase in which the fatty phase (wax(es)+oil(s)) is dispersed in the form of droplets so as to form a composition that is macroscopically homogeneous to the naked eye.

### Aqueous Phase

A composition according to the invention comprises water.

The term “aqueous phase” is intended to mean a phase comprising water and also optionally all the solvents and ingredients that are water-soluble or water-miscible (water-miscibility greater than 50% by weight at 25° C.), for instance lower monoalcohols containing from 1 to 5 carbon atoms such as ethanol or isopropanol, glycols containing from 3 to 8 carbon atoms such as propylene glycol, 1,3-butylene glycol, caprylyl glycol, pentylene glycol and dipropylene glycol.

The aqueous phase may contain a demineralized water or alternatively a floral water such as cornflower water and/or a mineral water such as Vittel water, Lucas water or La Roche Posay water and/or a spring water.

In particular, a composition according to the invention comprises at least 30.0% by weight, better still at least 40.0% by weight, or even a content of from 50% to 60% by weight of water, relative to the total weight of the composition.

### Fatty Phase

A composition according to the invention comprises at least one fatty phase containing:
i) at least one wax different from the C₄₀-C₄₈ monoesters of a saturated linear fatty acid;
ii) at least one non-polar non-volatile hydrocarbon oil; and
iii) at least one fatty alcohol.

The term “fatty phase” is intended to mean a phase comprising at least one fatty substance such as a wax, an oil, a pasty substance and also optionally all the solvents and ingredients that are soluble or miscible in said fatty phase (miscibility greater than 50% by weight at 25° C.). Such a fatty phase is a non-aqueous and water-immiscible organic phase.

### i) Waxes

The fatty phase in accordance with the invention comprises at least one wax.

The term “waxes” is intended to mean lipophilic compounds, which are solid at ambient temperature (25° C.) and at atmospheric pressure (760 mmHg), with a reversible solid/liquid change of state, which have a melting point of greater than or equal to 40° C., which may be up to 120° C.

For the purposes of the invention, the melting point corresponds to the temperature of the most endothermic peak observed on thermal analysis (DSC) as described in the standard ISO 11357-3; 1999. The melting point of the wax may be measured using a differential scanning calorimeter (DSC), for example the calorimeter sold under the name DSC Q2000 by the company TA Instruments.

The waxes used in the compositions of the invention are formed from complex mixtures and are notably described in the document Ullmann's Encyclopedia of Industrial Chemistry 2015, Wiley-VCH Verlag Gmbh & Co. KGaA.

The waxes in accordance with the invention in particular are different from the fatty alcohols iii) such as cetyl alcohol, stearyl alcohol or mixtures thereof, and also from the C₄₀-C₄₈ monoesters of a saturated linear fatty acid, and more particularly those corresponding to the following formula (1):

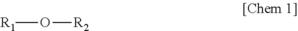

in which R¹ and R² are linear and saturated and have, independently of one another, a number of carbon atoms ranging from 20 to 24, with R₁ representing an acyl radical, and R² representing an alkyl radical, in particular chosen from arachidyl arachidate and behenyl behenate.

The waxes used in the compositions of the invention may notably be natural but also synthetic.

The term “natural” wax is understood to denote any wax pre-existing in nature or which can be transformed, extracted or purified from natural compounds existing in nature.

Among natural waxes, mention may notably be made of waxes termed fossil waxes, including those of petroleum origin, such as ozokerite, pyropissite, macrocrystalline waxes, also known as paraffins—including crude or gatsch waxes, gatsch raffinates, de-oiled gatsch, soft waxes, semirefined waxes, filtered waxes, refined waxes—and microcrystalline waxes, termed microwaxes, including bright stock gatsch. The fossil waxes also contain lignite, also referred to as montan wax, or peat wax.

As natural waxes other than fossil waxes, mention may be made of animal and plant waxes.

Mention may be made, as examples of vegetable waxes, of carnauba wax, candelilla wax, ouricury wax, sugarcane wax, jojoba wax, Trithrinax campestris wax, raffia wax, alfalfa wax, wax extracted from Douglas fir, sisal wax, flax wax, cotton wax, Batavia dammar wax, cereal wax, tea wax, coffee wax, rice wax, palm wax, Japan wax, their mixtures and their derivatives.

As examples of animal waxes, mention may be made of beeswax, Ghedda wax, shellac, Chinese wax, lanolin, also known as wool wax, mixtures thereof and derivatives thereof.

These waxes are generally multicomponent. For example, natural beeswax is approximately 70% composed of esters which are mainly monoesters (of fatty acid and of fatty alcohol), but also of hydroxy esters, of diesters and triesters and esters of sterols, and also of long-chain linear hydrocarbons, of free acids and of free alcohols. For obvious reasons, the weight proportion of their ingredients and their degree of purity are difficult to guarantee from one production batch to another.

The term “synthetic” wax is understood to denote waxes, the synthesis of which requires one or more chemical reactions carried out by a human being.

Among the synthetic waxes, semi-synthetic waxes and totally synthetic waxes can be distinguished. Synthetic waxes can be waxes obtained via a Fischer-Tropsch process, for example constituted of paraffins with a number of carbon atoms ranging from 20 to 50, or polyolefin waxes, for example homo- or copolymers of ethylene, of propene or of butene, indeed even of longer-chain α-olefins. The latter can be obtained by thermomechanical degradation of plastic polyethylene, by the Ziegler process, by high-pressure processes, or also via processes catalysed by metallocene entities. These waxes can be crystallizable, partially crystallizable or amorphous. The abovementioned synthetic waxes are generally non-polar and can be chemically treated in order to obtain polar waxes, for example by one or more of the following reactions: air oxidation, grafting, esterification, neutralization with metal soaps, amidation, direct copolymerizations or addition reactions.

According to a preferred embodiment, the composition according to the invention comprises at least one wax chosen from:
white beeswax with the INCI name: WHITE BEESWAX or CERA ALBA, for instance the commercial product CERABEIL LOR® sold by the company BAERLOCHER;
a synthetic beeswax with the INCI name: SYNTHETIC BEESWAX, for instance the commercial product CYCLOCHEM 330® from the company EVONIK GOLDSCHMIDT;
the carnauba wax with the INCI name: COPERNICIA CERIFERA (CARNAUBA) WAX/COPERNICIA CERIFERA CERA, for instance the product CERAUBA T1® sold by the company BAERLOCHER;
mixtures of paraffin waxes and hydrocarbon waxes with the INCI name PARAFFIN, for instance the commercial product SASOLWAX 5603@ sold by the company SASOL; and mixtures thereof.

The wax(es) in accordance with the invention may be present in a content of greater than or equal to 5% by weight relative to the total weight of the composition, better still greater than or equal to 7% by weight, or even greater than or equal to 10% by weight relative to the total weight of the composition. Preferably, it is (they are) present in a content ranging from 12% to 40% by weight relative to the total weight of the composition, better still from 14% to 35% and even better still from 16% to 30% by weight relative to the total weight of the composition.

### ii) Non-Polar Non-Volatile Hydrocarbon Oil

The oily phase in accordance with the invention comprises at least one non-polar non-volatile hydrocarbon oil.

The term “non-polar hydrocarbon oil” is understood to mean an oil containing exclusively hydrogen and carbon atoms.

The term “non-volatile oil” refers to an oil that remains on the skin or the keratin fibre at room temperature and atmospheric pressure for at least several hours, and that notably has a vapour pressure of less than 2.66 Pa, preferably less than 0.13 Pa. By way of example, the vapour pressure may be measured via the static method or via the effusion method by isothermal thermogravimetry, depending on the vapour pressure (standard OCDE 104).

As non-volatile hydrocarbon oil that can be used in the compositions of the invention, mention may be made of linear or branched hydrocarbons, of mineral or synthetic origin, such as:
liquid paraffins and derivatives thereof;
liquid petroleum jelly;
polydecenes, polybutenes; hydrogenated or non-hydrogenated polyisobutenes such as Parleam;
squalane,
mixtures thereof.

Use will more particularly be made of a polybutene such as the commercial product INDOPOL H 1500® from INEOS.

According to a preferred embodiment, the content of non-polar non-volatile hydrocarbon oil(s) in the composition in accordance with the invention ranges from 0.1% to 10% by weight, in particular from 1% to 5% by weight, and better still from 1.5% to 2% by weight, relative to the total weight of the composition.

iii) Fatty Alcohol

The oily phase in accordance with the invention comprises at least one fatty alcohol.

The fatty alcohol(s) is (are) in particular chosen from linear or branched, saturated or unsaturated C₁₄-C₃₀, preferably C₁₄-C₂₄, and even better still C₁₄-C₂₀, fatty alcohols.

According to a particularly preferred embodiment, the fatty alcohol is in the form of a mixture of several different fatty alcohols, and is preferably a mixture of several linear and saturated C₁₄-C₃₀, better still C₁₄-C₂₄, even better still C₁₄-C₂₀, fatty alcohols.

Preferably, the fatty alcohol according to the invention is chosen from cetyl alcohol (C₁₆), stearyl alcohol (C₁₈) and mixtures thereof (also known as “cetearyl alcohol”). Preferentially, the fatty alcohol according to the invention is a mixture of cetyl alcohol and stearyl alcohol. Such a mixture is notably sold under the name Lannette O OR/MBR by the company BASF.

According to a preferred embodiment, the fatty alcohol is solid at ambient temperature.

The fatty alcohol is present in the compositions of the invention in amounts ranging from 1.0% to 20.0% by weight, relative to the total weight of the composition, preferably from 2.0% to 15.0% by weight and even more particularly from 3.0% to 10.0% by weight, relative to the total weight of the composition.

C₁₂-C₂₀ Monoalkyl Phosphate

The composition of the invention comprises at least one anionic surfactant of C₁₂-C₂₀ monoalkyl phosphate type, partially or completely neutralized with a base.

The designation “monoalkyl” is intended to mean that the phosphate element is linked to a single C₁₂-C₂₀ alkyl chain, unless otherwise specified. The monoalkyl phosphate(s) (including phosphine oxide(s)) that may be used in the compositions according to the present application is/are chosen from C₁₄-C₂₀ and preferably C₁₆-C₁₈ monoalkyl phosphates and mixtures thereof.

Preferably, they are chosen from monocetyl phosphate, monostearyl phosphate and monocetearyl phosphate.

According to a first variant, the base is at least one organic base.

Preferably, the base of organic origin is chosen from amino acids such as arginine; alkanolamines such as monoethanolamine, diethanolamine, triethanolamine, triisopropanolamine, aminomethylpropanol; primary (poly) hydroxyalkylamines such as 2-amino-2-(hydroxymethyl) propane-1,3-diol (also known as tromethamine) and aminomethylpropanediol; and mixtures thereof.

According to a second variant, the base is at least one inorganic base.

This inorganic base is preferably chosen from alkali metal hydroxides and ammonium (NH₄⁺).

Preferably, the inorganic base is chosen from potassium hydroxide.

In particular, use will be made of the anionic surfactant in its form neutralized with potassium hydroxide (KOH), namely the potassium salt of monocetyl phosphate having the INCI name Potassium Cetyl Phosphate, for example sold under the names Amphisol K® and Amphisol A® from DSM Nutritional Products, Arlatone MAP® from Uniqema and Crodafos MCA® from Croda.

According to a preferred embodiment, the content of monoalkyl phosphate(s) ranges from 0.1% to 20% by weight, preferably from 0.5% to 12% by weight, relative to the total weight of the composition.

### Pullulan

The composition of the invention comprises at least one pullulan.

The pullulan in accordance with the invention is a polysaccharide constituted of maltotriose units, known under the name α-(1,4)-α(1,6)-glucan. Three glucose units in maltotriose are connected via an α-(1,4) glycoside bond, whereas the consecutive maltotriose units are connected to each other via an α-(1,6) glycoside bond. It is produced from starch by the fungus Aureobasidium pullulans. The pullulan is for example produced under the trade reference Pullulan PF 200 by the Hayashibara group in Japan or under the reference Aqua Beta® by the company Daiso Co. Ltd.

According to one particularly preferred embodiment of the invention, the pullulan is present in the composition in a content ranging from 1% to 7% by weight, preferably from 1% to 6% by weight, or even preferably from 2% to 5% by weight, relative to the total weight of the composition.

### Particles of Film-Forming Polymer in Suspension

The composition of the present invention comprises at least particles of film-forming polymer suspended in the aqueous phase.

In the present application, the term “film-forming polymer” means a polymer that is capable of forming, by itself or in the presence of an auxiliary film-forming agent, a continuous deposit on a support, at a temperature ranging from 20° C. to 150° C.

Said solid particles of film-forming polymer can either be used as is and are in suspension in the aqueous phase of the composition or can be used in the form of particles in aqueous dispersion.

According to one particular formula of the invention, the water-dispersible film-forming polymer may be in the form of an aqueous dispersion of particles generally bearing the name (pseudo) latex, i.e. latex or pseudolatex. Techniques for preparing these dispersions are well known to those skilled in the art.

The composition according to the invention may comprise one or more types of particle, these particles possibly varying as regards their size, their structure and/or their chemical nature.

The film-forming polymer suspended in the aqueous phase is preferably present in a solids content ranging from 1% to 20% by weight relative to the total weight of the composition, more preferentially ranging from 1% to 15% by weight, and more particularly ranging from 2% to 10% by weight relative to the total weight of the composition.

These solid particles may be of anionic, cationic or neutral nature and may constitute a mixture of solid particles of different natures.

Among the film-forming polymers that may be used in the composition of the present invention, mention may be made of synthetic polymers, of free-radical type or of polycondensate type, and polymers of natural origin, and mixtures thereof. In general, these polymers may be statistical polymers, block copolymers of A-B type, of A-B-A or else ABCD, etc. multiblock type, or even grafted polymers.

### a) Radical Film-Forming Polymer

The term “free-radical polymer” means a polymer obtained by polymerization of unsaturated and especially ethylenically unsaturated monomers, each monomer being capable of homopolymerizing (unlike polycondensates).

The film-forming polymers of free-radical type may notably be acrylic and/or vinyl homopolymers or copolymers.

The vinyl film-forming polymers can result from the polymerization of ethylenically unsaturated monomers having at least one acid group and/or of the esters of these acid monomers and/or of the amides of these acid monomers.

Ethylenically unsaturated monomers having at least one acid group or monomer bearing an acid group that may be used include α,β-ethylenic unsaturated carboxylic acids such as acrylic acid, methacrylic acid, crotonic acid, maleic acid or itaconic acid. (Meth)acrylic acid and crotonic acid are used in particular, and more particularly (meth)acrylic acid.

The esters of acid monomers are advantageously chosen from (meth)acrylic acid esters (also known as (meth)acrylates), notably (meth)acrylates of an alkyl, in particular of a C₁-C₂₀ and more particularly C₁-C₈ alkyl, (meth)acrylates of an aryl, in particular of a C₆-C₁₀ aryl, and (meth)acrylates of a hydroxyalkyl, in particular of a C₂-C₆ hydroxyalkyl.

Among the alkyl (meth)acrylates that may be mentioned are methyl methacrylate, ethyl methacrylate, butyl methacrylate, isobutyl methacrylate, 2-ethylhexyl methacrylate and lauryl methacrylate.

Among the hydroxyalkyl (meth)acrylates that may be mentioned are hydroxyethyl acrylate, 2-hydroxypropyl acrylate, hydroxyethyl methacrylate and 2-hydroxypropyl methacrylate.

Among the aryl (meth)acrylates, mention may be made of benzyl acrylate and phenyl acrylate.

The (meth)acrylic acid esters are in particular alkyl (meth)acrylates.

According to the present invention, the alkyl group of the esters may be either fluorinated or perfluorinated, i.e. some or all of the hydrogen atoms of the alkyl group are substituted with fluorine atoms.

Examples of amides of the acid monomers that may be mentioned are (meth)acrylamides, and especially N-alkyl (meth)acrylamides, in particular of a C₂-C₁₂ alkyl. Among the N-alkyl (meth)acrylamides that may be mentioned are N-ethylacrylamide, N-t-butylacrylamide and N-t-octylacrylamide.

The vinyl film-forming polymers may also result from the homopolymerization or copolymerization of monomers chosen from vinyl esters and styrene monomers. In particular, these monomers may be polymerized with acid monomers and/or esters thereof and/or amides thereof, such as those mentioned previously.

Examples of vinyl esters that may be mentioned are vinyl acetate, vinyl neodecanoate, vinyl pivalate, vinyl benzoate and vinyl t-butylbenzoate.

Styrene monomers that may be mentioned include styrene and α-methylstyrene.

The list of monomers given is not limiting, and it is possible to use any monomer known to those skilled in the art included in the categories of acrylic and vinyl monomers (including monomers modified with a silicone chain).

Mention may also be made of polymers resulting from free-radical polymerization of one or more free-radical monomers inside and/or partially at the surface of pre-existing particles of at least one polymer chosen from the group constituted of polyurethanes, polyureas, polyesters, polyesteramides and/or alkyds. These polymers are generally referred to as “hybrid polymers”.

### b) Polycondensate

As film-forming polymer of polycondensate type, mention may be made of anionic, cationic, nonionic or amphoteric polyurethanes, acrylic polyurethanes, polyvinylpyrrolidone-polyurethanes, polyester-polyurethanes, polyether-polyurethanes, polyureas, polyurea/polyurethanes and silicone polyurethanes, and mixtures thereof.

The film-forming polyurethane may be, for example, an aliphatic, cycloaliphatic or aromatic polyurethane, polyurea/urethane or polyurea copolymer including, alone or as a mixture, at least one block chosen from:
a block of aliphatic and/or cycloaliphatic and/or aromatic polyester origin, and/or
a branched or unbranched silicone block, for example polydimethylsiloxane or polymethylphenylsiloxane, and/or
a block comprising fluoro groups.

The film-forming polyurethanes as defined in the invention can also be obtained from branched or unbranched polyesters or from alkyds comprising mobile hydrogens, which are modified by reaction with a diisocyanate and a difunctional organic compound (for example dihydro, diamino or hydroxyamino), additionally comprising either a carboxylic acid or carboxylate group, or a sulfonic acid or sulfonate group, or alternatively a neutralizable tertiary amine group or a quaternary ammonium group.

Mention may also be made, among the film-forming polycondensates, of polyesters, polyesteramides, fatty-chain polyesters, polyamides and epoxyester resins.

The polyesters may be obtained, in a known manner, by polycondensation of dicarboxylic acids with polyols, especially diols.

The dicarboxylic acid may be aliphatic, alicyclic or aromatic. Examples of such acids that may be mentioned include: oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexanedicarboxylic acid, isophthalic acid, terephthalic acid, 2,5-norbornanedicarboxylic acid, diglycolic acid, thiodipropionic acid, 2,5-naphthalenedicarboxylic acid, 2,6-naphthalenedicarboxylic acid. These dicarboxylic acid monomers may be used alone or as a combination of at least two dicarboxylic acid monomers. Among these monomers, the ones chosen in particular are phthalic acid, isophthalic acid and terephthalic acid.

The diol can be chosen from aliphatic, alicyclic or aromatic diols. The diol used is chosen in particular from: ethylene glycol, diethylene glycol, triethylene glycol, 1,3-propanediol, cyclohexanedimethanol and 4-butanediol. Other polyols that may be used are glycerol, pentaerythritol, sorbitol and trimethylolpropane.

The polyesteramides may be obtained in a manner analogous to that of the polyesters, by polycondensation of diacids with diamines or amino alcohols. Diamines that may be used are ethylenediamine, hexamethylenediamine and meta- or para-phenylenediamine. An amino alcohol that may be used is monoethanolamine.

### c) Polymer of Natural Origin

Use may be made in the present invention of optionally modified polymers of natural origin, such as shellac resin, sandarac gum, dammar resins, elemi gums, copal resins, water-insoluble cellulose-based polymers such as nitrocellulose, modified cellulose esters especially including carboxyalkylcellulose esters such as those described in patent application US 2003/185774, and mixtures thereof.

According to a particular embodiment of the invention, said at least one film-forming polymer in the dispersed state is chosen from acrylic polymer dispersions, polyurethane dispersions, sulfopolyester dispersions, vinyl dispersions, polyvinyl acetate dispersions, vinylpyrrolidone, dimethylaminopropylmethacrylamide and lauryldimethylpropylmethacrylamidoammonium chloride terpolymer dispersions, dispersions of polyurethane/polyacrylic hybrid polymers and dispersions of particles of core-shell type, and mixtures thereof.

Various types of aqueous dispersions, notably commercial aqueous dispersions, which are suitable for the preparation of the composition in accordance with the present invention, are described in detail below.

Thus, according to a preferred embodiment of the invention, the aqueous dispersion of polymer particles is an aqueous dispersion of acrylic polymer.

The acrylic polymer may be a styrene/acrylate copolymer and notably a polymer chosen from copolymers resulting from the polymerization of at least one styrene monomer and at least one C₁-C₁₈ alkyl (meth)acrylate monomer. As styrene monomers that may be used in the invention, examples that may be mentioned include styrene and α-methylstyrene, and in particular styrene. The C₁-C₁₈ alkyl (meth)acrylate monomer is in particular a C₁-C₁₂ alkyl (meth)acrylate and more particularly a C₁-C₁₀ alkyl (meth)acrylate. The C₁-C₁₈ alkyl (meth)acrylate monomer may be chosen from methyl acrylate, methyl methacrylate, ethyl acrylate, propyl acrylate, butyl acrylate, butyl methacrylate, hexyl acrylate, octyl acrylate, 2-ethylhexyl acrylate, lauryl (meth)acrylate and stearyl (meth)acrylate.

As acrylic polymer in aqueous dispersion, use may be made according to the invention of:
aqueous dispersions of acrylic polymer with the INCI name ACRYLATES COPOLYMER such as the products sold under the names Epitex 66 Copolymer® by Dow Chemical, Acronal DS-6250@ by BASF, Neocryl A-45®, Neocryl XK-90®, Neocryl A-1070®, Neocryl A-1090®, Neocryl BT-62®, Neocryl A-1079® and Neocryl A-523® by DSM, Joncryl 95®, Joncryl 8211@ by BASF, Daitosol 5000 AD®;
aqueous dispersions of acrylic polymer with the INCI name ACRYLATES/ETHYLHEXYL ACRYLATE COPOLYMER such as the product sold under the name Daitosol 5000 SJ® by DAITO KASEY KOGYO;
aqueous dispersions of acrylic polymer with the INCI name STYRENE/ACRYLATES/AMMONIUM METHACRYLATE such as the product sold under the name Syntran 5760 CG® by Interpolymer.

According to another alternative embodiment of the invention, the aqueous dispersion of polymer particles is an aqueous dispersion of particles of polyester-polyurethane and/or polyether-polyurethane which is in particular anionic. The anionic nature of the polyester-polyurethanes and of the polyether-polyurethanes used according to the invention is due to the presence in their constituent units of groups bearing a carboxylic acid or sulfonic acid function. The polyester-polyurethane or polyether-polyurethane particles used according to the invention are generally sold in aqueous dispersion form.

The particle content of said dispersions currently available on the market ranges from about 20% to about 50% by weight relative to the total weight of the dispersion.

Among the anionic polyester-polyurethane dispersions that may be used in the aqueous varnishes according to the invention, mention may be made in particular of the product sold under the name Avalure UR 405® by Noveon.

Among the anionic polyether-polyurethane particle dispersions that may be used according to the invention, mention may be made in particular of the products sold under the name Avalure UR 450® by Noveon and under the name Neorez R 970® by DSM.

According to a particular embodiment of the invention, use may be made of a mixture of commercial dispersions constituted of anionic polyester-polyurethane particles as defined above and of anionic polyether-polyurethane particles also defined above.

For example, use may be made of a mixture constituted of the dispersion sold under the name Sancure 861® or a mixture of the dispersion sold under the name Avalure UR 405® and of the dispersion sold under the name Avalure UR 450®, these dispersions being sold by Noveon.

As solid particles of film-forming polymer according to the invention, use may be made more preferentially of:
aqueous dispersions of acrylic polymer (INCI name: ACRYLATES COPOLYMER) which are sold under the names Epitex 66 Copolymer® by Dow Chemical, Acronal DS-6250® by BASF, Neocryl A-45®, Neocryl XK-90®, Neocryl A-1070®, Neocryl A-1090®, Neocryl BT-62®, Neocryl A-1079® and Neocryl A-523® by DSM, Joncryl 95@ or Joncryl 8211® by BASF, Daitosol 5000 AD® or Daitosol 5000 SJ® (INCI name: Acrylates/Ethylhexyl Acrylate Copolymer) by Daito Kasei Kogyo; Syntran 5760 CG® (INCI name: STYRENE/ACRYLATES/AMMONIUM METHACRYLATE COPOLYMER) by Interpolymer,
the aqueous polyurethane dispersions sold under the names Neorez R-981® and Neorez R-974® by DSM, Avalure UR-405®, Avalure UR-410®, Avalure UR-425®, Avalure UR-450®, Sancure 875®, Avalure UR 445® and Avalure UR 450® by Noveon and Impranil 85 by Bayer,
sulfopolyesters such as those sold under the brand name Eastman AQ® by Eastman Chemical Products,
vinyl dispersions, such as Mexomere PAM®, aqueous dispersions of polyvinyl acetate, such as Vinybran® from Nisshin Chemical or those sold by Union Carbide, aqueous dispersions of vinylpyrrolidone, dimethylaminopropylmethacrylamide and lauryldimethylpropylmethacrylamidoammonium chloride terpolymer, such as Styleze W® from ISP
aqueous dispersions of polyurethane/polyacrylic hybrid polymer such as the products sold under the references Hybridur® by Air Products or Duromer® from National Starch.

According to a preferred embodiment, a composition in accordance with the invention is an aqueous dispersion of acrylic film-forming polymer with the INCI name: ACRYLATES COPOLYMER such as the commercial product Epitex 66 Copolymer® sold by Dow Chemical.

### Pigment

A composition according to the invention, and notably those intended for makeup, generally comprises at least one pigment.

The term “pigment” means a pulverulent material, also known as a particulate material, formed from white or coloured, mineral or organic particles, which are insoluble in an aqueous medium, and which are intended to colour and/or opacify the resulting composition and/or film. These pigments may be white or coloured, and mineral and/or organic.

The pigments may be white or coloured, mineral and/or organic, and coated or uncoated. Among the mineral pigments that may be mentioned are titanium dioxide, optionally surface-treated, zirconium oxide, zinc oxide or cerium oxide, and also iron oxide, chromium oxide, manganese violet, ultramarine blue, chromium hydrate and ferric blue. Among the organic pigments that may be mentioned are carbon black, pigments of D & C type and lakes based on cochineal carmine or on barium, strontium, calcium or aluminium.

The pigments may be pearlescent agents, notably chosen from white pearlescent pigments, such as mica covered with titanium or with bismuth oxychloride, coloured pearlescent pigments, such as titanium mica with iron oxides, titanium mica with notably ferric blue or chromium oxide, or titanium mica with an organic pigment of the abovementioned type, and also pearlescent pigments based on bismuth oxychloride.

Preferably, the pigment is chosen from metal oxides, notably titanium dioxides, iron oxides and mixtures thereof; and more particularly black iron oxides (CI 77499).

Preferably, the pigment(s) is (are) present in the composition in a content ranging from 2.0% to 25.0% by weight, preferably from 3.0% to 20.0% by weight, more particularly from 4.0% to 15.0% by weight, relative to the total weight of the composition.

### Other Components

### I/ Additional Water-Soluble Film-Forming Polymers

According to one particular embodiment, the composition according to the invention may further contain at least one additional water-soluble film-forming polymer, i.e. other than the pullulan.

As examples of water-soluble film-forming polymers, mention may be made of:
proteins, for instance proteins of plant origin such as wheat or soybean proteins; proteins of animal origin such as keratins, for example keratin hydrolysates and sulfonic keratins;
cellulose polymers such as hydroxyethylcellulose, hydroxypropylcellulose, methylcellulose, ethylhydroxyethylcellulose and carboxymethylcellulose, and also quaternized cellulose derivatives;
acrylic polymers or copolymers, such as polyacrylates or polymethacrylates;
vinyl polymers, for instance polyvinylpyrrolidones, copolymers of methyl vinyl ether and of malic anhydride, the copolymer of vinyl acetate and of crotonic acid, copolymers of vinylpyrrolidone and of vinyl acetate; copolymers of vinylpyrrolidone and of caprolactam; polyvinyl alcohol;
anionic, cationic, amphoteric or nonionic chitin or chitosan polymers;
acacia gums, guar gum, xanthan derivatives, karaya gum;
alginates and carrageenans;
glycosaminoglycans, hyaluronic acid and derivatives thereof;
shellac resin, sandarac gum, dammar resins, elemi gums, copal resins;
deoxyribonucleic acid;
mucopolysaccharides such as chondroitin sulfates;
mixtures thereof.

Preferentially, an additional water-soluble film-forming polymer is chosen from cellulose polymers, acacia gums and mixtures thereof.

More particularly, a mixture of hydroxyethylcellulose and acacia gum will be used.

According to a preferred embodiment of the invention, the composition comprises less than 5.0% by weight of additional water-soluble film-forming polymer(s), more preferentially less than 2.0% by weight, relative to the total weight of the composition.

### II/ Nonionic Surfactants

According to one particular embodiment, the composition according to the invention may further comprise at least one nonionic surfactant.

Within the meaning of the present invention, a “surfactant” is understood to mean an amphiphilic surfactant compound, i.e. one which has two parts of different polarity. Generally, one is lipophilic (soluble or dispersible in an oily phase). The other is hydrophilic (soluble or dispersible in water). Surfactants are characterized by the value of their HLB (hydrophilic-lipophilic balance), the HLB being the ratio of the hydrophilic part to the lipophilic part in the molecule. The term “HLB” is well known to those skilled in the art and is described, for example, in “The HLB System. A Time-Saving Guide to Emulsifier Selection” (published by ICI Americas Inc.; 1984). The HLB of the surfactant(s) used according to the invention can be determined by the Griffin method or the Davies method.

For the purposes of the present invention, a “nonionic surfactant” is understood to mean any surfactant having no ionic (anionic or catatonic) charge in its structure.

According to a preferred embodiment of the invention, the composition comprises less than 5.0% by weight of nonionic surfactant(s), more preferentially less than 2.0% by weight, relative to the total weight of the composition.

According to a particularly preferred embodiment, the nonionic surfactant(s) is (are) chosen from polyoxyethylenated alcohols, which may comprise from 2 to 200 oxyethylene units, preferably from 2 to 30 oxyethylene units, in particular polyoxyethylenated fatty alcohols, notably C₈-C₂₄, and preferably C₁₂-C₁₈ fatty alcohols, such as Steareth-2, for instance the product SP BRIJ S2 MBAL-SO-(AP)® sold by CRODA, Steareth-20, for instance the product SP BRIJ S20 SEG-PA-(RB)® sold by CRODA, and Ceteareth-30, for instance the product EUMULGIN B 3® sold by BASF.

In particular, a mixture of Steareth-2 and Steareth-20 will be used.

### III/ Pasty Fatty Substance

According to one particular embodiment, the composition according to the invention may further comprise at least one pasty fatty substance.

For the purposes of the present invention, the term “pasty fatty substance” is intended to mean a lipophilic fatty compound with a reversible solid/liquid change of state, which comprises, at a temperature of 23° C., a liquid fraction and a solid fraction.

In other words, the initial melting point of the pasty compound may be below 23° C. The liquid fraction of the pasty compound measured at 23° C. may represent 9% to 97% by weight of the compound. This fraction that is liquid at 23° C. preferably represents between 15% and 85% and more preferably between 40% and 85% by weight.

Preferably, the pasty fatty substance(s) has (have) a final melting point below 60° C.

Preferably, the pasty fatty substance(s) has (have) a hardness of less than or equal to 6 MPa.

Preferably, the pasty fatty substance(s) has (have), in the solid state, a crystalline organization, which is visible by X-ray diffraction characterization.

For the purposes of the invention, the melting point corresponds to the temperature of the most endothermic peak observed on thermal analysis (DSC) as described in the standard ISO 11357-3; 1999. The melting point of a pasty substance or of a wax may be measured using a differential scanning calorimeter (DSC), for example the calorimeter sold under the name DSC Q2000 by the company TA Instruments.

As regards the measurement of the melting point and the determination of the final melting point, the sample preparation and measurement protocols are as follows:

A sample of 5 mg of pasty fatty substance, preheated to 80° C. and withdrawn with magnetic stirring using a spatula that is also heated, is placed in a hermetic aluminium capsule, or a crucible. Two tests are performed to ensure the reproducibility of the results.

The measurements are performed on the abovementioned calorimeter. The oven is flushed with nitrogen. Cooling is performed by an RCS 90 heat exchanger. The sample is then subjected to the following protocol: it is first placed at a temperature of 20° C., and then subjected to a first temperature rise passing from 20° C. to 80° C., at a heating rate of 5° C./minute, then is cooled from 80° C. to −80° C. at a cooling rate of 5° C./minute and finally subjected to a second temperature rise passing from −80° C. to 80° C. at a heating rate of 5° C./minute. During the second temperature rise, the variation in the difference between the power absorbed by the empty crucible and by the crucible containing the sample of pasty substance or wax as a function of the temperature is measured. The melting point of the compound is the temperature value corresponding to the top of the peak of the curve representing the variation in the difference in power absorbed as a function of the temperature.

The final melting point corresponds to the temperature at which 95% of the sample has melted.

The liquid fraction by weight of the pasty compound at 23° C. is equal to the ratio of the heat of fusion consumed at 23° C. to the heat of fusion of the pasty compound.

The heat of fusion of the pasty compound is the heat consumed by the compound in order to pass from the solid state to the liquid state. The pasty compound is said to be in the solid state when all of its mass is in crystalline solid form. The pasty compound is said to be in the liquid state when all of its mass is in liquid form.

The heat of fusion of the pasty compound is equal to the integral of the entire melting curve obtained using the abovementioned calorimeter, with a temperature rise of 5° C. or 10° C. per minute, according to the standard ISO 11357-3:1999. The heat of fusion of the pasty compound is the amount of energy required to make the compound change from the solid state to the liquid state. It is expressed in J/g.

The heat of fusion consumed at 23° C. is the amount of energy absorbed by the sample to change from the solid state to the state that it has at 23° C., constituted of a liquid fraction and a solid fraction.

The liquid fraction of the pasty compound measured at 32° C. preferably represents from 30% to 100% by weight of the compound, preferably from 50% to 100%, more preferably from 60% to 100%, by weight of the compound. When the liquid fraction of the pasty compound measured at 32° C. is equal to 100%, the temperature of the end of the melting range of the pasty compound is below or equal to 32° C.

The liquid fraction of the pasty compound measured at 32° C. is equal to the ratio of the heat of fusion consumed at 32° C. to the heat of fusion of the pasty compound. The heat of fusion consumed at 32° C. is calculated in the same way as the heat of fusion consumed at 23° C.

As regards the measurement of the hardness, the sample preparation and measurement protocols are as follows:

The pasty fatty substance is placed in a mould 75 mm in diameter, which is filled to approximately 75% of its height. In order to overcome the thermal history and to control the crystallization, the mould is placed in a Votsch VC0018 programmable oven, where it is first placed at a temperature of 80° C. for 60 minutes, then cooled from 80° C. to 0° C. at a cooling rate of 5° C./minute, and then left at the stabilized temperature of 0° C. for 60 minutes, and then subjected to a temperature rise ranging from 0° C. to 20° C., at a heating rate of 5° C./minute, and then left at the stabilized temperature of 20° C. for 180 minutes.

The compression force measurement is taken using a TA/TX2i texturometer from Swantech.

The spindle used is chosen according to the texture:
cylindrical steel spindle 2 mm in diameter for starting materials which are very rigid;
cylindrical steel spindle 12 mm in diameter for starting materials which are not very rigid.

The measurement comprises three steps: a first step after automatic detection of the surface of the sample, where the spindle moves at a measuring speed of 0.1 mm/s, and penetrates into the pasty fatty substance to a penetration depth of 0.3 mm, the software notes the maximum force value reached; a second “relaxation” step where the spindle remains at this position for one second and the force is noted after 1 second of relaxation; finally, a third “withdrawal” step in which the spindle returns to its initial position at a speed of 1 mm/s, and the probe withdrawal energy (negative force) is noted.

The hardness value measured during the first step corresponds to the maximum compression force measured in newtons divided by the area of the texturometer cylinder expressed in mm² in contact with the pasty fatty substance. The hardness value obtained is expressed in megapascals or MPa.

### Polyol-Fatty Acid (Poly)Ester

According to one particular embodiment, the composition in accordance with the invention comprises at least one pasty fatty substance (C1) chosen from the polyol-fatty acid (poly)esters.

The composition according to the invention comprises at least one (poly)ester of at least one fatty acid comprising from 20 to 100 carbon atoms and of at least one polyol.

According to the invention, the polyol-fatty acid (poly)ester may be a monoester, diester or triester, or even a mixture thereof. Preferably, the fatty acid is chosen from fatty acids in which the linear or branched carbon chain comprises from 20 to 50, preferably from 20 to 30 and preferentially from 20 to 25 carbon atoms. According to an advantageous embodiment, the fatty acid according to the invention is chosen from C₂₂ fatty acids, and is preferentially behenic acid.

According to the invention, the polyol may notably be chosen from diols. The diol can be chosen from aliphatic, alicyclic or aromatic diols. The diol used is preferably chosen from: ethylene glycol, diethylene glycol, triethylene glycol, 1,3-propanediol, cyclohexanedimethanol and 4-butanediol. Other polyols that may be used include glycerol, pentaerythritol, sorbitol and trimethylolpropane.

According to an advantageous embodiment, the polyol according to the invention is glycerol.

According to one embodiment, the composition according to the invention comprises at least one pasty fatty substance (C1) chosen from (poly)esters of behenic acid and glycerol.

Thus, the compound (C1) may for example be chosen from mixtures of monoesters, diesters and/or triesters of C₂₂ carboxylic acid(s) and glycerol. The compound (C1) or ester of behenic acid and glycerol is notably chosen from the group constituted of glyceryl monobehenate, glyceryl dibehenate, glyceryl tribehenate and mixtures thereof. The glycerol-carboxylic acid ester can be in particular a mixture of glyceryl monobehenate, glyceryl dibehenate and glyceryl tribehenate.

Such a mixture is notably available, for example, under the INCI name GLYCERYL DIBEHENATE & TRIBEHENIN & GLYCERYL BEHENATE and is notably sold under the reference COMPRITOL 888® by GATTEFOSSE. It is also present as a mixture in the starting material bearing the INCI name TRIBEHENIN PEG-20 ESTER sold under the reference EMULIUMO 22® by GATTEFOSSE.

Preferably, the content of pasty fatty substance (C1) in a composition according to the invention is greater than or equal to 0.1%, preferably ranging from 0.5% to 5%, and preferably from 1% to 3%, by weight relative to the total weight of said composition.

### Additional Pasty Fatty Substance (C2)

The composition according to the invention may further comprise at least one additional pasty fatty substance (C2), i.e. other than (C1).

Among the additional pasty fatty substances, mention may for example be made of synthetic compounds and compounds of plant origin. Such a pasty fatty substance may be obtained by synthesis from starting materials of plant origin.

This (these) pasty fatty substance(s) (C2) is (are) advantageously chosen from:
lanolin and derivatives thereof;
liquid petroleum jelly, in particular the product for which the INCI name is PETROLATUM and which is sold under the name ULTIMA WHITE PET USP® by Penreco;
polyol ethers chosen from ethers of pentaerythritol and of polyalkylene glycol, ethers of fatty alcohol and of sugar, and mixtures thereof, the polyethylene glycol pentaerythrityl ether comprising 5 oxyethylene units (5 OE) (CTFA name: PEG-5 Pentaerythrityl Ether), the polypropylene glycol pentaerythrityl ether comprising 5 oxypropylene units (5 OP) (CTFA name: PPG-5 Pentaerythrityl Ether), and mixtures thereof, and more especially the PEG-5 Pentaerythrityl Ether, PPG-5 Pentaerythrityl Ether and soybean oil mixture sold under the Lanolide name by Vevy, which is a mixture in which the constituents are in a 46/46/8 ratio by weight: 46% PEG-5 Pentaerythrityl Ether, 46% PPG-5 Pentaerythrityl Ether and 8% soybean oil;
hydrogenated oils, notably hydrogenated palm oil and hydrogenated jojoba oil;
polymeric or non-polymeric silicone compounds;
polymeric or non-polymeric fluorinated compounds;
vinyl polymers, notably:
olefin homopolymers and copolymers,
hydrogenated diene homopolymers and copolymers,
linear or branched homopolymer or copolymer oligomers of alkyl (meth)acrylates preferably containing a C₈-C₃₀ alkyl group,
homopolymer or copolymer oligomers of vinyl esters containing C₈-C₃₀ alkyl groups, homopolymer or copolymer oligomers of vinyl ethers containing C₈-C₃₀ alkyl groups,
liposoluble polyethers resulting from polyetherification between one or more C₂-C₁₀₀, preferably C₂-C₅₀ diols,
esters other than the abovementioned C₁ compounds,
and/or mixtures thereof.

The composition according to the invention may comprise one or more additional pasty fatty substances (C2) in a total content of greater than or equal to 0.01% by weight, relative to the total weight of the composition, preferably between 0.1% and 5% by weight, even better still from 0.1% to 2% by weight, relative to the total weight of the composition.

According to one embodiment, a composition according to the invention comprises at least one additional pasty fatty substance (C2) chosen from hydrogenated oils, notably hydrogenated palm oil and hydrogenated jojoba oil, esters of a glycerol oligomer, and mixture(s) thereof.

According to a particular embodiment, a composition according to the invention comprises at least one additional pasty fatty substance (C2) chosen from esters of a glycerol oligomer, notably in a content greater than or equal to 0.1%, preferably between 0.5% and 5%, and preferentially between 1% and 3%, by weight relative to the total weight of said composition.

The esters of a glycerol oligomer are notably chosen from diglycerol esters, in particular condensates of adipic acid and of glycerol, for which some of the hydroxyl groups of the glycerols have reacted with a mixture of fatty acids such as stearic acid, capric acid, stearic acid and isostearic acid, and 12-hydroxystearic acid, such as the one with the INCI name BIS-DIGLYCERYL POLYACYLADIPATE-2, for example sold under the brand name Softisan 649@ by Sasol.

According to a particularly preferred embodiment, a composition according to the invention comprises a mixture comprising
a) at least one pasty fatty substance (C1) which is a mixture of glyceryl monobehenate, glyceryl dibehenate and glyceryl tribehenate; and
b) at least one pasty fatty substance (C2) which is a mixture of hydrogenated jojoba oil, hydrogenated palm oil and diglyceryl oligomer ester with the INCI name BIS-DIGLYCERYL POLYACYLADIPATE-2.

### Additives

In addition to the abovementioned compounds, a composition according to the invention may of course comprise one or more cosmetic additives usually used, notably chosen from preserving agents, bactericidal agents, antioxidants, fragrances, lipophilic active agents, hydrophilic active agents, liposoluble dyes, moisturizers, emollients, sunscreens, and mixtures thereof.

Needless to say, a person skilled in the art will take care to select the optional additional additives and/or the amount thereof such that the advantageous properties of the composition according to the invention are not, or are not substantially, adversely affected by the envisaged addition.

### Physical Characteristics

### a) Solids Content

The composition according to the invention advantageously has a solids content of at least 42.0% by weight, and preferentially of at least 44.0% by weight, relative to the total weight of the composition, or even from 45.0% to 60.0% by weight, relative to the total weight of the composition.

For the purposes of the present invention, the term “solids content” denotes the content of nonvolatile matter.

The amount of solids content (abbreviated as SC) of a composition according to the invention is measured using a Halogen Moisture Analyzer HR 73® commercial halogen desiccator from Mettler Toledo. The measurement is performed on the basis of the weight loss of a sample dried by halogen heating, and thus represents the percentage of residual matter once the water and the volatile matter have evaporated off.

This technique is fully described in the machine documentation supplied by Mettler Toledo®.

The measuring protocol is as follows:

Approximately 2 g of the composition, referred to hereinbelow as the sample, are spread out on a metal crucible, which is placed in the halogen desiccator mentioned above. The sample is then subjected to a temperature of 105° C. until a constant weight is obtained. The wet mass of the sample, corresponding to its initial mass, and the dry mass of the sample, corresponding to its mass after halogen heating, are measured using a precision balance.

The experimental error associated with the measurement is of the order of plus or minus 2%.

The solids content is calculated in the following manner:

$$Solids content ( expressed as \% by weight ) = 100 \times \frac{dry weight}{wet weight} \qquad [ Math 1 ]$$

The composition may be manufactured via the known processes generally used in the cosmetics field.

The composition used according to the invention may be a makeup composition, a makeup base, notably for keratin fibres, or base coat, a composition to be applied onto makeup, also known as topcoat, or else a composition for treating keratin fibres.

More especially, the composition according to the invention is a mascara.

Such compositions are notably prepared according to the general knowledge of those skilled in the art.

### Packaging and Application Assembly or Kit

The present invention also relates to an assembly, or kit, for packaging and applying a cosmetic composition for caring for and/or making up keratin materials, comprising:
a packaging device comprising the cosmetic composition as described above,
an applicator for said composition.

Said applicator may be integrally attached to a gripping member forming a cap for said packaging device. In other words, said applicator may be mounted in a removable position on said device between a closed position and an open position of a dispensing aperture of the device for packaging said composition.

An assembly for coating keratin materials suitable for the invention may comprise an applicator configured for applying said cosmetic composition for coating keratin fibres and, where appropriate, a packaging device suitable for receiving said composition.

### Applicator

The applicator may comprise means for smoothing and/or separating keratin fibres, such as the eyelashes or the eyebrows, notably in the form of teeth, bristles, spikes or other reliefs.

The applicator is arranged to apply the composition to the eyelashes or the eyebrows, and may comprise, for example, a brush or a comb.

The applicator may also be used for finishing of the makeup, over a region of the eyelashes or eyebrows that is made up or laden with the composition.

The brush may comprise a twisted core and bristles held between the turns of the core, or may be made in yet another way.

The comb is, for example, produced from a single part by moulding of a plastic.

In certain exemplary embodiments, the application element is mounted at the end of a stem, which stem may be flexible, which may contribute to improving the comfort during application.

### Packaging Device

The packaging device comprises a container intended for housing the composition for caring for and/or making up keratin materials. This composition may then be withdrawn from the container by immersing the applicator therein.

This applicator may be firmly attached to an element for closing the container. This closing element may form a member for gripping the applicator. This gripping member may form a cap to be removably mounted on said container by any suitable means, such as by screwing, click-fastening, coupling, etc. Such a container may thus reversibly house said applicator.

This container may be optionally equipped with a wiper suitable for removing surplus product taken up by the applicator.

A method for applying the composition according to the invention to the eyelashes or the eyebrows may also include the following steps:
forming a deposit of the cosmetic composition on the eyelashes or the eyebrows,
leaving the deposit on the eyelashes or the eyebrows, it being possible for the deposit to dry.

It should be noted that, according to another embodiment, the applicator may form a product container. In such a case, a container may, for example, be provided in the gripping member and an internal channel can internally connect this gripping member to the application elements in relief.

Finally, it should be noted that the packaging and application assembly may be in the form of a kit, it being possible for the applicator and the packaging device to be housed separately in the same packaging article.

The expressions “of between . . . and . . . ” and “ranging from . . . to . . . ” should be understood as meaning limits included, unless otherwise specified.

In the description and the examples, the percentages are weight percentages, unless otherwise indicated. The percentages are thus expressed by weight relative to the total weight of the composition. The ingredients are mixed in the order and under the conditions that are readily determined by a person skilled in the art.

The invention will now be described by means of examples which are present for purely illustrative purposes and should not be interpreted as examples that limit the invention.

### EXAMPLES 1, 1A, 1B: MASCARAS

The following compositions were prepared.

TABLE 1

| | | | 1a | 1b |
|---|---|---|---|---|
| | | | (outside | (outside |
| | | 1 | the | the |
| Phases | Ingredients | (invention) | invention) | invention) |
| A1 | WHITE BEESWAX/CERA ALBA | 2.2 | 2.2 | 2.2 |
| | (CERABEIL LOR ®-BAERLOCHER) | | | |
| | SYNTHETIC BEESWAX | 2.2 | 2.2 | 2.2 |
| | (CYCLOCHEM 330 ®-EVONIK | | | |
| | GOLDSCHMIDT) | | | |
| | COPERNICIA CERIFERA (CARNAUBA) | 3.5 | 3.5 | 3.5 |
| | WAX/COPERNICIA CERIFERA CERA | | | |
| | (CERAUBA T1 ®- BAERLOCHER) | | | |
| | PARAFFIN | 8.1 | 8.1 | 8.1 |
| | (SASOLWAX 5603 ®-SASOL) | | | |
| | STEARETH-2 | 1.0 | 1.0 | 1.0 |
| | (SP BRIJ S2 MBAL-SO-(AP) ®-CRODA) | | | |
| | STEARETH-20 | 1.1 | 1.1 | 1.1 |
| | (SP BRIJ S20 SEG-PA-(RB) ®-CRODA) | | | |
| | CETYL ALCOHOL | 2.0 | 2.0 | — |
| | (LANETTE 16 ®-BASF) | | | |
| | HYDROGENATED JOJOBA OIL | 0.25 | 0.25 | 0.25 |
| | (JOJOBA WAX FLAKES ®-DESERT | | | |
| | WHALE) | | | |
| | HYDROGENATED PALM OIL | 0.25 | 0.25 | 0.25 |
| | (GV 60/SG ®-SIO) | | | |
| A2 | GLYCERYL DIBEHENATE (and) | 2.0 | 2.0 | 2.0 |
| | TRIBEHENIN (and) GLYCERYL BEHENATE | | | |
| | (COMPRITOL 888 ®-GATTEFOSSE) | | | |
| A3 | POLYBUTENE | 2.0 | 2.0 | 2.0 |
| | (INDOPOL H 1500 ®-Ineos) | | | |
| A4 | BIS-DIGLYCERYL POLYACYLADIPATE-2 | 2.0 | 2.0 | 2.0 |
| | (SOFTISAN 649 ®-CREMER OLEO) | | | |
| B1 | WATER/AQUA | q.s. for 100 | q.s. for 100 | q.s. for 100 |
| B2 | CAPRYLYL GLYCOL | 0.3 | 0.3 | 0.3 |
| | SODIUM DEHYDROACETATE | 0.3 | 0.3 | 0.3 |
| | PHENOXYETHANOL | 0.8 | 0.8 | 0.8 |
| B3 | PULLULAN | 3.0 | — | 3.0 |
| | (PULLULAN COSMETIC GRADE ® | | | |
| | HAYASHIBARA) | | | |
| | ACACIA SENEGAL GUM | 0.63 | 0.63 | 0.63 |
| | HYDROXYETHYLCELLULOSE | 0.75 | 0.75 | 0.75 |
| | (CELLOSIZE HEC QP-4400H EUROPE | | | |
| | PCG ®-AMERCHOL) | | | |
| B4 | IRON OXIDES | 7.0 | 7.0 | 7.0 |
| | (CI 77499) | | | |
| | (SUNPURO C33-700 ® from SUN) | | | |
| B5 | POTASSIUM CETYL PHOSPHATE | 7.0 | 7.0 | 7.0 |
| | (AMPHISOL K ®-DSM NUTRITIONAL | | | |
| | PRODUCTS) | | | |
| C | Aqueous dispersion containing 45% by | 7.0 | 7.0 | 7.0 |
| | weight of ACRYLATES COPOLYMER | (3.15% | (3.15% | (3.15% |
| | film-forming polymer | active | active | active |
| | (EPITEX 66 POLYMER ®-DOW | material) | material) | material) |
| | CHEMICAL) | | | |

### Protocol for Preparing Examples 1 to 3

### Preparation of the Fatty Phase

All the starting materials used were carefully weighed out using a balance (accuracy 0.01 g).

The various waxes and the waxy surfactants (steareth-2, steareth-20 and cetyl alcohol) were melted in a 500 ml jacketed heating pan with circulation of hot oil to control the temperature. The mixture was heated to about 96-98° C.

Once the waxes had melted, they were homogenized by stirring using a Moritz blender, which is stirring of rotor-stator type: this machine is constituted of a fixed part in which a second mobile part rotates at variable speed, and is used for making emulsions since it can produce a very high shear.

When the waxes and surfactants have been melted and homogenized, the black pigments were added and everything is then homogenized again in the Moritz blender. The temperature was then checked (by means of a probe) and adjusted to 95-96° C. if necessary.

### Preparation of the Aqueous Phase

A tall-form 600 ml beaker was used, which was first weighed empty in order to adjust the mass of water before emulsification. The amount of water required, preheated by an electric kettle, was stirred slowly in this beaker using a Rayneri blender.

A Rayneri blender is a laboratory stirrer equipped with a blade or turbine for stirring at variable speed while constituents are being incorporated.

Still with slow stirring, the following were introduced successively: the latex and then the potassium cetyl phosphate (surfactant). Between each introduction, care was taken to ensure good dissolution of the compound and homogenization of the medium.

When all the compounds were dissolved, it was necessary to adjust the mass of water in order to compensate for the evaporation that has taken place. Sodium dehydroacetate antifoaming agent and caprylyl glycol were then added.

The aqueous phase was then placed in a water bath (nominal temperature 90-92° C.) until a temperature of 88-90° C. was reached.

### Emulsification

When the two phases were at the desired temperature, the aqueous phase was added very slowly into the fatty phase while gradually increasing the stirring; the phase inversion was then visible as a change in viscosity. The stirring was adjusted to the maximum for 10 minutes (around 3500 rpm), to avoid overflowing if the stirring is too vigorous.

### Temperature Reduction

After the emulsification, the heating pan was placed under a Rayneri blender then equipped with a butterfly blade, which enabled blending and homogenization during the temperature reduction, at low shear. The stirring speed was low so as not to incorporate air bubbles.

By means of the oil bath, the temperature is reduced gradually: in 10° C. steps, homogenizing thoroughly at each step, especially at about 70° C.

The mascara thus obtained was transferred into a closed jar to prevent it from drying out on contact with air; it was then necessary to wait 24 hours to check the homogeneity of the formulation and the correct dispersion of the pigments.

### Black Intensity Measurement

L* colorimetric measurements were carried out on each example of composition by spreading the compositions on respective FAP H247G® glass dishes covered with a microscope slide and by evaluating the colour obtained with said compositions using a Konica Minolta spectrocolorimeter associated with SpectraMagic® software measuring the SCI and SCE values. 3 measurements were taken in a triangle for each example 1, 1a and 1b.

In an L* a* b* system, the lower the value of L*, the more intense the colour, and in particular the darker the colour.

The measurement of L* was carried out in the bulk.

Next, ΔL* corresponding to the difference in the L* value of composition 1 according to the invention minus the L* value of comparative composition 1a or 1b is calculated. When ΔL* is greater than 1, it is considered that the deviation is significant between the L* value of composition 1 according to the invention and the L* value of composition 1a or 1b.

### Colorimetric Results:

TABLE 1

| In the bulk | L* | AL* |
|---|---|---|
| Example 1 according to | 14.61 ± 0.0829 | / |
| them invention | | |
| Comparative example 1a | 16.038 ± 0.0891 | 1.428 |
| Comparative example 1b | 15.77 ± 0.072 | 1.160 |

The results obtained showed that Example 1 according to the invention produced the most intense colour compared with Examples 1a and 1b outside the invention.

1. Composition for caring for and/or making up keratin materials, such as the skin, notably the contour of the eyes, the contour of the eyelashes, the contour of the eyebrows; keratin fibres such as the eyelashes and the eyebrows, in the form of an emulsion, in particular wax(es)-in-water emulsion, comprising, preferably in a physiologically acceptable medium:
a) an aqueous phase, and
b) at least one oily phase dispersed in the aqueous phase comprising
i) at least one wax; and
ii) at least one non-polar non-volatile hydrocarbon oil; and
iii) at least one fatty alcohol; and
c) at least one C₁₂-C₂₀ monoalkyl phosphate, partially or completely neutralized with a base; and
d) at least one pullulan; and
e) particles of film-forming polymer suspended in the aqueous phase; and
f) at least one pigment.

2. Composition according to claim 1, comprising at least 30.0% by weight of water, relative to the total weight of the composition.

3. Composition according to claim 1, wherein the at least one wax is different from the at least one fatty alcohol and is not a C40-C48 monoester of a saturated linear fatty acid.

4. Composition according to claim 1, wherein the at least one wax is chosen from:
white beeswax;
synthetic beeswax;
mixtures of paraffin waxes and hydrocarbon waxes; and mixtures thereof.

5. Composition according to claim 1, wherein the wax(es) is (are) present in a content of greater than or equal to 5% by weight relative to the total weight of the composition.

6. Composition according to claim 1, wherein the wax(es) is (are) present in a content from 12% to 40% by weight relative to the total weight of the composition.

7. Composition according to claim 1, wherein the at least one non-polar non-volatile hydrocarbon oil is chosen from linear or branched hydrocarbons, of mineral or synthetic origin, chosen from:
liquid paraffins;
liquid petroleum jelly;
polydecenes, polybutenes; hydrogenated or non-hydrogenated polyisobutenes;
squalane, and
mixtures thereof.

8. Composition according to claim 1, wherein the at least one non-polar non-volatile hydrocarbon oil is a polybutene.

9. Composition according to claim 1, wherein the content of non-polar non-volatile hydrocarbon oil(s) ranges is from 0.1% to 10% by weight relative to the total weight of the composition.

10. Composition according to claim 1, wherein the at least one fatty alcohol is chosen from linear or branched, saturated or unsaturated, C₁₄-C₃₀, fatty alcohols.

11. Composition according to claim 1, wherein the at least one fatty alcohol is present in amounts from 1.0% to 20.0% by weight, relative to the total weight of the composition.

12. Composition according to claim 1, wherein the at least one C₁₂-C₂₀ monoalkyl phosphate, partially or completely neutralized with a base is the potassium salt of monocetyl phosphate.

13. Composition according to claim 1, wherein the content of C₁₂-C₂₀ monoalkyl phosphate(s) is from 0.1% to 20% by weight relative to the total weight of the composition.

14. Composition according to claim 1, wherein the at least one pullulan is present in the composition in a content from 1% to 7% by weight relative to the total weight of the composition.

15. Composition according to claim 1, wherein the particles of film-forming polymer suspended in the aqueous phase is present in a solids content from 1% to 20% by weight relative to the total weight of the composition.

16. Composition according to claim 1, wherein the particles of film-forming polymer suspended in the aqueous phase is an aqueous dispersion of acrylic polymer.

17. Composition according to claim 1, wherein the at least one pigment is chosen from metal oxides, iron oxides and mixtures thereof.

18. Composition according to claim 1, wherein the pigment(s) is (are) present in the composition in a content ranging from 2.0% to 25.0% by weight relative to the total weight of the composition.

19. Composition according to claim 1, further comprising at least one additional water-soluble film-forming polymer.

20. Composition according to claim 19, comprising less than 5.0% by weight of additional water-soluble film-forming polymer(s) relative to the total weight of the composition.

21. Composition according to claim 1, further comprising at least one nonionic surfactant.

22. Composition according to claim 21, comprising less than 5.0% by weight of nonionic surfactant(s) relative to the total weight of the composition.

23. Composition according to claim 1, further comprising at least one pasty fatty substance.

24. Composition according to claim 23, comprising said at least one pasty fatty substance in a content of greater than or equal to 0.01% by weight, relative to the total weight of the composition.

25. (canceled)

26. (canceled)

27. Composition according to claim 23, comprising a mixture comprising
a) at least one pasty fatty substance (C1) which is a mixture of glyceryl monobehenate, glyceryl dibehenate and glyceryl tribehenate; and
b) at least one pasty fatty substance (C2) which is a mixture of hydrogenated jojoba oil, hydrogenated palm oil and diglyceryl ester.

28. Composition according to claim 1, in the form of a mascara.

29. Assembly, or kit, for packaging and applying a cosmetic composition for coating keratin materials, comprising:
a packaging device comprising the composition as defined in claim 1;
an applicator for said composition.

30. Method for coating keratin materials, in particular the skin, notably the contour of the eyes, of the eyelashes or of the eyebrows; keratin fibres such as the eyelashes and/or the eyebrows, comprising a step of applying to said keratin materials at least one composition as defined according to claim 1.
